(19) United States
(12) Patent Application Publication
(10) Pub. No.: US 2021/0187242 A1
(43) Pub. Date: Jun. 24, 2021

(54) Fluid Delivery System for Creating Separation Between Biological Surfaces

(71) Applicant: Ethicon, Inc., Somerville, NJ (US)

(72) Inventors: William Cohn, Houston, TX (US); Nicolo Garbin, Houston, TX (US); Matthew Kuhn, Houston, TX (US); Sharma Tushar, Houston, TX (US)

(21) Appl. No.: 16/724,451

(22) Filed: Dec. 23, 2019

(51) Int. Cl. A61M 25/01; A61M 25/00

(52) U.S. Cl. CPC A61M 25/0108; A61M 25/0084; A61M 25/0075; A61B 2562/0271; A61M 2025/0002; A61M 2210/125; A61M 2205/3337; A61M 2205/3334; A61M 2205/3368; A61M 2202/0225; A61M 2210/105; A61M 2230/65

(57) ABSTRACT

A system comprising: a fluid supply configured for the delivery of a fluid; a hollow body in fluid-communication with the fluid supply, the hollow body configured to be disposed between a first biological surface and a second biological surface; and a mechanism configured to control delivery of fluid from the fluid supply and through the hollow body to create separation between the first biological surface and the second biological surface.

### BACKGROUND

### 1. Field of the Present Disclosure

The present disclosure relates to systems, methods and devices for preventing esophageal damage, and more particularly to systems, methods and devices for preventing esophageal damage after catheter ablation.

### 2. Discussion of the Related Art

Cardiac arrhythmias, and atrial fibrillation, persist as common and dangerous medical ailments, especially in the aging population. In patients with normal sinus rhythm, the heart, which is comprised of atrial, ventricular, and excitatory conduction tissue, is electrically excited to beat in a synchronous, patterned fashion. In patients with cardiac arrhythmias, abnormal regions of cardiac tissue do not follow the synchronous beating cycle associated with normally conductive tissue as in patients with normal sinus rhythm. Instead, the abnormal regions of cardiac tissue aberrantly conduct to adjacent tissue, thereby disrupting the cardiac cycle into an asynchronous cardiac rhythm. Such abnormal conduction has been previously known to occur at various regions of the heart, for example, in the region of the sino-atrial (SA) node, along the conduction pathways of the atrioventricular (AV) node and the Bundle of His, or in the cardiac muscle tissue forming the walls of the ventricular and atrial cardiac chambers.

Atrial fibrillation affects millions of Americans. Patients with atrial fibrillation have a significantly increased risk of suffering from a stroke, heart attack, and other adverse events. Catheter ablation has emerged as a dominant therapy for treating atrial fibrillation. By creating full-thickness lines of scar tissue in the left atrium, the chaotic waves of electrical activity necessary to maintain atrial fibrillation are isolated, and the patient's heart rhythm converts to a regular organized one. The lines of scar tissue must be full-thickness, which is to say, must extend from the inner lining of the heart, the endocardium, all the way through the entire thickness of the atrial wall to the outer lining, the epicardium. If the scar tissue is only partial-thickness, the electrical waves can still propagate around the scar.

Biosense Webster is a global leader in the field of treating atrial fibrillation. The Biosense Webster CARTO® 3 system allows accurate mapping of the atrium, navigation inside the atrium with an ablation catheter, and creation of full-thickness lesions. Despite the sophistication of the Biosense Webster system, avoiding esophageal damage and occasional post procedural development of an atrial-esophageal fistula remains a challenge. This complication occurs because of the proximity between the esophagus, the swallowing tube that connects the mouth or more accurately, the pharynx to the stomach, and the back wall of the left atrium.

When creating the pattern of left atrial scar that has been identified as most effective in converting atrial fibrillation, it is often necessary to create a line that runs transversely across the back wall of the left atrium. During the ablation procedure, the esophagus may likely be damaged from the conduction of thermal energy. Even in pulmonary vein isolation ablation procedures, in which a transverse line across the back wall of the left atrium is not created, the esophagus is frequently damaged due to its proximity to cardiac structures. This is particularly challenging because there is no evidence during the procedure that suggests the esophagus has been injured unless a temperature sensor(s) is placed into the esophagus to notify the physician that esophageal tissue damage is occurring. Furthermore, temperature sensing devices placed into the esophagus are not preventative and provide no protective benefit to the esophagus. As a result, the esophagus is often damaged during an ablation procedures and, in some cases, causes the formation of an atrial-esophageal fistula. The classic presentation of this complication is that of a patient who returns two weeks after an ablation procedure with a low-grade fever of unknown origin or a small stroke. On further investigation, it is revealed that the patient has developed endocarditis, an infection of the heart and heart lining, resulting from drainage of esophageal contents into the heart, or that the patient has had a stroke which resulted from a small bubble of air arising from the esophageal lumen that has passed into the left atrium. Regardless of presentation, the development of an atrial esophageal fistula or abnormal passageway is a serious and often deadly complication. Patients generally must undergo a major thoracic operation if crisis is to be averted, and even with early surgical intervention, the majority of these patients ultimately die. Because of increased awareness of this complication, physicians less aggressively ablate tissue is in close proximity with the esophagus. Catheter ablation for converting atrial fibrillation to normal organized rhythm requires the successful creation of full-thickness lines of scar tissue in a prescribed pattern throughout the left atrium. If the burns do not involve the full thickness of the left atrium wall, the therapy is unlikely to be successful. Electric current may still travel through the partial thickness of living heart muscle and atrial fibrillation persists. As a consequence of less-aggressively ablating the heart wall in the attempt of minimizing esophageal damage, lines of scar tissue in the left atrium often fail to extend through the full thickness of the heart wall, and fewer patients benefit from successful conversion to regular rhythm as a result.

There is consensus among electrophysiologists that a solution is needed to allow aggressive treatment of the left atrium without risk of this potential complication.

Others have proposed solutions. The two main types are: 1) devices that utilize a shaped balloon, rod, or nitinol structure in an effort to pull the esophagus away from the back wall of the left atrium so the electrophysiologist can be more aggressive creating posterior burns; or 2) devices passed down the esophagus that measure temperature, impedance, or other metrics to inform the electrophysiologist when it is safe to burn and when it is not, or when the esophagus is heating up during ablation so the electrophysiologist can stop immediately.

The challenges with the first type include the need for the electrophysiologist to manipulate the esophagus, something with which they typically have little familiarity, and the challenges with moving the esophagus. The two structures, the esophagus and the left atrium, are immediately adjacent to each other in an air-tight space. As one attempts to pull the esophagus away from the left atrium, the atrium is pulled somewhat in conjunction with the esophagus. Moreover, there have been reports of esophageal injury while trying to pull the esophagus by applying traction to it from within its lumen. These injuries include occasional esophageal hematomas and perforations, which may require surgical treatment. These devices fail to create true separation between these two structures, and instead often involve moving part of the esophagus laterally away from the left atrium. The esophagus remains in-contact with left atrium and can still be unintentionally burned.

The challenges with esophageal temperature monitoring center around its reactive nature. This monitoring only allows the electrophysiologist to determine that the esophagus lumen has increased in temperature, indicating that a thermal insult to the esophageal wall has already occurred. Although this measurement allows the electrophysiologist to immediately stop burning and in so doing, limit the extent of the thermal exposure, the measurement does nothing to prevent such injury from happening.

Accordingly, there exists a need for a reliable system, method and device for preventing esophageal damage and fistula formation during catheter ablation of the left atrium.

### SUMMARY OF THE DISCLOSURE

The present disclosure relates to system(s), method(s) and device(s) for creating separation between biological surfaces such as tissues, tissue planes, and/or organs, for example, using carbon dioxide for various clinical applications. Such applications may include thermal protection of the esophagus, avoiding mechanical/thermal damage of an underlying tissue during dissection by creating separation of the tissue planes with CO2, or protecting against radiation enteropathy via creation of a radiation-impermeable layer of hydrogel/CO2). Other applications may benefit from the present disclosure.

A system may comprise a fluid supply configured for the delivery of a fluid; a hollow body in fluid-communication with the fluid supply, the hollow body configured to be disposed between a first biological surface and a second biological surface; and a mechanism configured to control delivery of fluid from the fluid supply and through the hollow body to create separation between the first biological surface and the second biological surface.

A system may comprise a hollow body configured to access a target location; a control element configured to control the delivery of a fluid through the hollow body; a sensor device configured to measure a parameter of the fluid flowing through the hollow body, wherein the parameter is used to determine at least an environment of the hollow body such that the hollow body may be moved to the target location in response to one or more of the parameter or changes to the parameter.

A system may comprise a hollow body configured to access a target location; a control element configured to actuate the delivery of a fluid through the hollow body; a sensor device configured to measure a parameter of the fluid flowing through the hollow body, wherein the parameter is used to actuate a flow of the fluid through the hollow body in response to one or more of the parameter or changes to the parameter.

A device may comprise a hollow body configured for the delivery of a fluid between a first biological surface and a second biological surface; and an anchoring mechanism configured to releasably secure the device to one or more of the first biological surface or the second biological surface.

Additionally or alternatively, the present disclosure relates to access routes and methods for delivering carbon dioxide to create separation between the esophagus and heart wall

A method may comprise: delivering a hollow body into the heart; advancing at least a portion of the hollow body through the heart wall; delivering a volume of fluid through the hollow body to create separation between the esophagus and the heart wall; and removing the hollow body after the delivery of fluid.

A method may comprise: delivering a hollow body into the esophagus; advancing at least a portion of the hollow body through the esophageal wall; delivering a volume of fluid through the hollow body to create separation between the esophagus and the heart wall; and removing the hollow body after the delivery of fluid.

A method may comprise: advancing at least a portion of a hollow body percutaneously into the patient's body; delivering a volume of fluid through the hollow body to create separation between the esophagus and the heart wall; and removing the hollow body after the delivery of fluid.

A method may comprise: delivering a hollow body into the airway; advancing at least a portion of the hollow body through the wall of the trachea; delivering a volume of fluid through the hollow body to create separation between the esophagus and the heart wall; and removing the hollow body after the delivery of fluid.

As a non-limiting example, the present disclosure is directed to system(s), method(s) and device(s) wherein sufficient volumes of carbon dioxide gas is injected between the esophagus and the back wall of the left atrium to create a protective layer of insulation that will prevent thermal injury to the esophagus while intentionally creating full-thickness burns in the left atrium. The present disclosure may overcome a number of the limitations associated with the prior art as briefly described above.

In accordance with one aspect, the present disclosure is directed to a catheter assembly for the delivery of gas to the fibro-fatty tissue between the esophagus and the heart for the prevention of esophageal damage and/or fistula during catheter ablation of the left atrium. The catheter assembly comprising a handle assembly including electronics and mechanical structures for the controlled delivery of medical grade gas supplied from a gas supply system, an anchoring assembly for positioning and securing the device in the proper position for the infusion of the gas into a predetermined location within the fibro-fatty tissue, the anchoring assembly including an inflatable/deflatable device with a deployable injection needle, and a catheter shaft interconnecting the handle assembly to the anchoring assembly and configured for delivery to a predetermined location within the human anatomy and including mechanical and electrical interconnections for modulation of the inflatable/deflatable anchoring mechanism, for the controlled delivery of gas via user input, and monitoring capability.

Carbon dioxide insufflation creates an insulating sleeve around the esophagus, in effect isolating the esophagus from the heart wall. The reference “Anatomic Relations Between the Esophagus and Left Atrium and Relevance for Ablation of Atrial Fibrillation,” Circulation 2005; 112:1400-1405, describes the heterogeneity with respect to the amount and thickness of fibro-fatty tissue interposed between the esophagus and the left atrium. In almost half of the cadavers they dissected, the thickness is less than 5 mm. When carbon dioxide is injected into this fibro-fatty layer, the tissue inflates, and becomes “emphysematous,” a term that describes solid tissue infused with gas. Trapped gas is an excellent insulator.

In accordance with the present disclosure, an insulative fluid (e.g., carbon dioxide) is delivered between the heart wall and the esophagus a layer of insulation surrounding the esophagus and providing adequate thermal insulation, thereby preventing esophageal injury during catheter ablation. Carbon dioxide is utilized instead of air to leverage carbon dioxide's water solubility. Carbon dioxide is very soluble in water, and in other fluids such as blood, and readily dissolves into solution when introduced into a blood vessel, making emboli formation highly unlikely. It is important to note that dosages of carbon dioxide less than 3 mL/kg per minute that has been introduced into the cranial circulatory system is tolerated with no neurotoxicity, but the potential to cause embolic stroke in the cranial system does exist (https://www.ncbi.nlm.nih.gov/pmc/articles/PMC4603680/). Because a gas is being injected, the needle to be utilized may be small enough, e.g. on the order of a 27-gauge needle, so that the risk of potential injury to the left atrium or esophagus is essentially non-existent.

### BRIEF DESCRIPTION OF THE DRAWINGS

The foregoing and other features and advantages of the disclosure will be apparent from the following, more particular description of preferred embodiments of the disclosure, as illustrated in the accompanying drawings.

FIG. 1 is a block diagram representation of an exemplary system for the prevention of esophageal damage and/or fistula formation during ablation in accordance with the present disclosure.

FIG. 2 is a representation of an exemplary transesophageal catheter assembly in accordance with the present disclosure.

FIG. 2A is a diagrammatic representation of the internal components of an exemplary transesophageal catheter handle in accordance with the present disclosure.

FIG. 2B is a diagrammatic representation of the connection points of the proximal end of an exemplary transesophageal catheter handle in accordance with the present disclosure.

FIG. 3A is a diagrammatic representation of an exemplary transesophageal catheter with an inflated balloon and undeployed needle in accordance with the present disclosure.

FIG. 3B is a diagrammatic representation of an exemplary transesophageal catheter with an inflated balloon and deployed needle in accordance with the present disclosure.

FIG. 3C is a diagrammatic representation of an exemplary transesophageal catheter with sensors integrated into the surface of a balloon in accordance with the present disclosure.

FIG. 3D is a diagrammatic representation of an exemplary transesophageal catheter with anti-slip grips on the external surface of the balloon in the form of ribs, spikes, pyramids, bumps, villi or similar protrusions in accordance with the present disclosure.

FIG. 3E is a diagrammatic representation of an exemplary transesophageal catheter with a three-dimensional position sensor positioned near the distal aspect of the catheter.

FIGS. 4A and 4B are diagrammatic representations of the needle slider mechanism of an exemplary transesophageal catheter in accordance with the present disclosure.

FIGS. 5A through 5D are diagrammatic representations of the distal aspect of another exemplary transesophageal catheter with a nitinol wire anchoring mechanism and undeployed needle in accordance with the present disclosure.

FIG. 6A is a diagrammatic representation of the distal aspect of another exemplary transesophageal catheter with an undeployed needle in accordance with the present disclosure.

FIG. 6B is a diagrammatic representation of the distal aspect of another exemplary transesophageal catheter with a deployed needle in accordance with the present disclosure.

FIG. 7 is a diagrammatic representation of another exemplary transesophageal catheter with temperature sensors integrated into the shaft of the catheter and a three-dimensional position sensor positioned near the distal aspect of the catheter in accordance with the present disclosure.

FIG. 8 is an alternative exemplary transesophageal catheter with an expanding nitinol cage anchoring mechanism in accordance with the present disclosure.

FIG. 9 is an alternative exemplary transesophageal catheter with an expanding asymmetrical balloon anchoring mechanism in accordance with the present disclosure.

FIGS. 10A-10D are diagrammatic representations of an exemplary gas delivery system in accordance with the present disclosure.

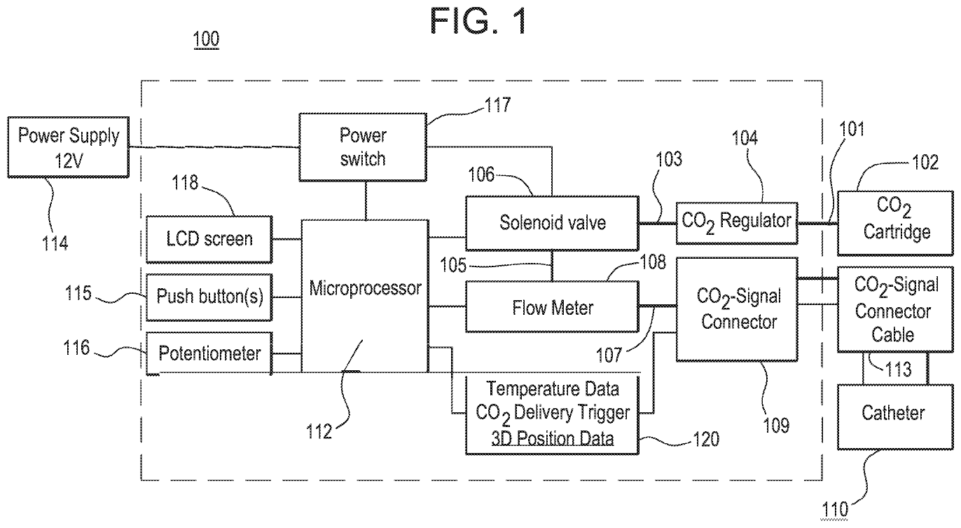

FIG. 1 is a graphical representation of flow rate versus needle penetration depth for various regions of the anatomy in accordance with the present disclosure.

FIG. 12 is a graphical representation of voltage versus needle penetration depth for various regions of the anatomy in accordance with the present disclosure.

FIG. 13 is a flow diagram of the insufflation process in accordance with the present disclosure.

### DETAILED DESCRIPTION OF THE PREFERRED EMBODIMENTS

The present disclosure relates to system(s), method(s) and device(s) for creating separation between biological surfaces such as tissues, tissue planes, and/or organs, for example, using carbon dioxide for various clinical applications. Such applications may include thermal protection of the esophagus, avoiding mechanical/thermal damage of an underlying tissue during dissection by creating separation of the tissue planes with CO2, or protecting against radiation enteropathy via creation of a radiation-impermeable layer of hydrogel/CO2). Other applications may benefit from the present disclosure.

Although various applications may benefit from the present disclosure, an illustrative example may comprise system(s), method(s) and device(s) for preventing or minimizing the formation of an esophageal fistula or esophageal tissue damage due to unintended thermal dispersion during ablation of the heart wall. In the present disclosure, carbon dioxide is injected or infused into the fibro-fatty tissue that separates the heart wall from the esophagus to expand the tissue and create an insulation layer therebetween. With the carbon dioxide infused tissue insulation layer in place, catheter ablation may be utilized to create full-thickness scar tissue with minimal risk of damaging the esophagus and forming an esophageal fistula. A description of experiments given below demonstrate the feasibility and efficacy of the inventive concept.

An eight-animal study was conducted to demonstrate that carbon dioxide could be safely injected through a catheter inserted up the femoral vein to the right atrium and through the right atrial wall into the pericardium to facilitate obtaining pericardial access. The study demonstrated that carbon dioxide may be safely injected into biological tissue. The study also demonstrated that carbon dioxide offers a number of advantages over air, including high solubility, low viscosity, radio-translucency and excellent thermal and electrical insulation qualities. More specifically, carbon dioxide which is fifty-four times more soluble than nitrogen and twenty-eight times more soluble than oxygen, is typically reabsorbed in less than two hours and is highly unlikely to result in gas embolus, even in large quantities, due to its solubility in water. Carbon dioxide has a low viscosity, allowing it to pass through a needle as small as a 33-gauge needle. The puncture from this size needle seals almost immediately after removal, even in the presence of systemic heparin, thereby reducing the likelihood of complications. Carbon dioxide is also visible under X-ray fluoroscopy, thereby allowing for visible confirmation of successful insufflation by creating an outline of the esophagus under X-ray fluoroscopy. Finally, carbon dioxide is a good electrical and thermal insulator which is exactly what is required to protect the esophagus during catheter ablation.

The eight-animal study was followed with two separate acute animal experiments. In each, the esophagus of a pig was exposed through a left thoracotomy. Because the esophagus does not run behind the left atrium in pigs, it was possible to directly observe the periesophageal tissue as an indicator of the feasibility of carbon dioxide injection to create a protective barrier layer. A carbon dioxide source was connected to a stopcock which allowed a 60-cc syringe connected to a 27-gauge needle to be filled with pure carbon dioxide. The carbon dioxide was injected into the soft tissue surrounding the esophagus. The carbon dioxide immediately dissected through the soft tissue surrounding the esophagus and increased the thickness of the fibro-fatty layer by creating an emphysema (carbon dioxide infused tissue). The carbon dioxide infused through the tissue all the way around the circumference of the esophagus and tracked toward the head and tail as far as the esophagus was exposed. The thickness of the barrier layer was demonstrated by cutting therethrough. The thickness of the gas-infused tissue was visible on X-ray, presenting as a lucent halo around the esophagus. One may also appreciate that the esophagus moved away from the spine due to the circumferential nature of the carbon dioxide emphysema. Essentially, the carbon dioxide emphysema isolates the esophagus from all other anatomical structures.

Upon completion of the pig studies, two human cadaver studies were conducted to demonstrate the feasibility of forming an insulation layer around the esophagus by creating an emphysema. In both cadavers, a simple investigation was conducted by injecting 120 cc (two complete 60 cc syringes) of carbon dioxide through the back wall of the left atrium. This was also done under direct vision, as the heart in each of the cadavers had been dissected. This study was an endeavor to demonstrate the feasibility of the concept of forming an insulation layer by creating an emphysema or separation. After cutting through the posterior left atrium wall, it was observed that emphysematous tissue between the left atrium and the esophagus formed as it did in the animal studies utilizing carbon dioxide.

The animal experiments were then repeated with additional steps. An esophageal temperature probe was utilized to monitor tissue temperature while intentionally creating lesions on the outer surface of the esophagus using an ablation catheter. Ablation of the esophageal wall was performed both with carbon dioxide insufflation and without carbon dioxide insufflation, to learn of the effects carbon dioxide has on the conduction of thermal energy.

In these evaluations, a multi-pole temperature probe was placed through the pig's mouth and down the esophagus under X-ray guidance. The ablation catheter was applied directly to the outer surface of the esophagus and the ablation electrode was aligned with one of the twelve (12) poles of the temperature sensor by X-ray. The ablation catheter was then energized. The measured temperature began to climb almost immediately, from a baseline temperature of 36.6 degrees C. With continued energy application, the temperature rose to 40 degrees C. after thirty (30) seconds. The experiment was then repeated under the same conditions, with the only difference being carbon dioxide insufflation was added to the protocol as is explained in greater detail subsequently.

Prior to infusing carbon dioxide to test thermal insulation of the esophagus during ablation, an investigation into how long carbon dioxide would remain in place after injection into the periesophageal space was performed. After injecting 120 cc of carbon dioxide into the periesophageal fibro-fatty tissue, the tissue would instantly inflate with carbon dioxide, becoming considerably thicker. Yet, the tissue would gradually return to baseline geometry within an hour. From this simple test it may be reasonably inferred that continuous insufflation with carbon dioxide would be preferable to insuring the insulating layer remained in place when needed during the ablation procedure.

Based on this observation, a 27-gauge needle attached to a long intravenous extension tube was attached directly to the regulator of a small tank of pressurized carbon dioxide. When the needle was inserted into the fibro-fatty tissue around the esophagus, it immediately inflated, as had been previously observed. But the cavity remained inflated until the supply of carbon dioxide was stopped. The rate of carbon dioxide delivery was arbitrarily titrated to be as low as possible with the regulator at hand.

When this experiment was repeated with an ablation catheter and a temperature probe (once again aligning the electrode with the temperature sensor under X-ray) and performing the ablation burn at the same power settings, the temperature readings were significantly different from those observed prior to infusion of carbon dioxide. After thirty (30) seconds of continuous burning, the temperature rose only 0.1 degrees C., from 36.6 degrees C. to 36.7 degrees C., in contrast to the 3.4 degrees C. observed when there was no carbon dioxide present; namely, 36.6 degrees to 40.0 degrees C. Accordingly, carbon dioxide injected into the fatty tissue surrounding the esophagus provided thermal insulation to the esophagus during such a procedure.

Dissection of the periesophageal tissue after only 120 cc of carbon dioxide injection or infusion reveals an 8 mm sheath or layer of emphysematous tissue that circumferentially surrounded the esophagus. This tissue is gas infused and poorly conducts radio frequency energy and heat. This 8 mm layer should push the posterior left atrium wall and the esophagus away from each other, thereby allowing aggressive burns to be created across the posterior left atrium wall without fear of esophageal injury.

A system for performing this procedure should preferably be simple for the electrophysiologist to utilize and not interfere with the underlying catheter ablation procedure. The system should preferably remain in position during the ablation and cause no injury to the left atrium, the esophagus or any biological tissue. The system may also counter the effects of systemic carbon dioxide absorption by utilizing a feedback controller to deliver additional carbon dioxide as needed to maintain the required tissue separation. The system may include a temperature probe. Initially, doctors may place a temperature probe in the esophagus to ensure that the carbon dioxide infused tissue does create a thermal barrier. Once enough evidence exists that proves that the esophagus is thermally insulated, the temperature probe may not be needed. The system may also be utilized just once at the onset of the catheter ablation procedure to achieve the desired separation between the esophagus and the left atrium and then subsequently removed to allow for the remainder of the ablation procedure, provided the effects of carbon dioxide absorption are negligible.

Referring now to FIG. 1, there is illustrated an exemplary embodiment of a fluid (e.g., gas) delivery system 100 for use in conjunction with the aforementioned transesophageal catheter for the prevention of esophageal damage and/or fistula during catheter ablation in accordance with the present disclosure. Although reference is made to catheter ablation applications, other processes may be used. The system 100 is configured to deliver carbon dioxide through a minimally invasive transesophageal catheter to create a gaseous pocket between the posterior wall of the left atrium and the esophagus. This pocket serves to thermally isolate and separate the esophagus from the left atrium during ablation to prevent esophageal damage or the formation of an atrial-esophageal fistula. It is important to note that the system 100 may be implemented utilizing a combination of discrete components, as a unitary, integrated system and/or a combination thereof.

The system 100 is configured as a closed-loop feedback control system and is illustrated in block diagram format for ease of explanation. Carbon dioxide, purified for use in biological applications, is supplied from a pressurized canister 102 and routed through a conduit 101 to a pressure regulator 104. As set forth above, special connectors may be utilized to prevent gas supplies other than carbon dioxide from being utilized. Although illustrated as a single discrete carbon dioxide canister, the gas may be supplied from any suitable source, for example, a central supply. In addition, the pressure regulator 104 may be connected directly to the pressurized canister 102. The pressure regulator 104 is adjustable, through manual or electronic means, and is utilized to set and maintain the pressure at which the carbon dioxide is delivered. The operation of the pressure regulator 104 is the same as a pressure regulator on a SCUBA tank or home compressor. A pressure regulator simply maintains the pressure of the gas to be released at a set value for downstream use. The pressure regulator 104 is connected to a solenoid-controlled valve 106 through conduit 103. The solenoid-controlled valve 106 is utilized to control the flow rate of the carbon dioxide from the canister 102 or other supply. The solenoid-controlled valve 106 is connected to a flowmeter 108 via conduit 105. The flowmeter 108 measures the flow rate of the carbon dioxide exiting the solenoid-controlled valve 106 to ensure that it is at the desired flow rate for use in the procedure. The flowmeter 108 is connected to a combination gas line and electronic signal connector 109 via conduit 107. The combination gas line and electronic signal connector 109 allows for delivery of carbon dioxide from the electronic gas delivery system 100 to the transesophageal catheter 110 as well as the transmission of temperature readings and three-dimensional (3D) position data from the transesophageal catheter 110 to the electronic gas delivery system 100 through the CO2-Signal connector cable 113. The transesophageal catheter 110 is utilized to precisely deliver the carbon dioxide to the desired location within the body as described herein. The conduits 101, 103, 105 and 107 may comprise any suitable material that does not react with carbon dioxide, for example, metallic materials such as stainless steel and polymeric materials such as polysiloxanes.

The system 100 also comprises a microprocessor or microcontroller 112. The microprocessor or microcontroller 112 is powered by a power supply 114. The power supply 114 may comprise a battery, either a primary battery or a secondary battery, and/or circuitry for converting power supplied from another source, for example, house power, into a voltage and current level suitable for the microprocessor 112 and other components of the system 100. The power supply 114 is connected to a power switch 117. The microprocessor 112 is programmed to read signals from the flowmeter 108 and the catheter 110 based upon feedback signals from each as well as preprogrammed control parameters. The microprocessor 112 also outputs control signals to the pressure regulator 104 to adjust the pressure of the gas as required, and to the solenoid-controlled valve 106 to precisely control, actuate, and regulate the delivery of a given volume of carbon dioxide gas. The user control push button(s) 115 are configured to allow the user of the system 100, for example a physician or electrophysiologist, to control the delivery of carbon dioxide gas (i.e. on/off control), deliver a pre-set volume of carbon dioxide (i.e. deliver 500 mL of carbon dioxide), cease the delivery of carbon dioxide (i.e. stopping the delivery of gas before the user-set volume has been reached), and to reset the measurement of the total amount of gas delivered. The user is able to pre-set a desired volume of gas to be delivered via the potentiometer 116. The microprocessor 112 modulates the parameters of operation via its connection to the solenoid-controlled valve 106 and operates as part of the feed-forward path of the control loop. The microprocessor 112, through its feedback control process automatically adjusts and maintains the operation of the system 100 in accordance with the user's settings. The microprocessor 112 may comprise any suitable processor and associated software and memory to implement the operation of the system 100. The microprocessor 112 continuously outputs the real-time reading of volumetric gas flow from the flow meter 108, the total amount of gas delivered, and the user pre-set volume to an LCD display 118 for ease of monitoring.

The microprocessor 112 also is in communication with a data unit 120. The data unit 120 communicates information/data between the microprocessor 112 and the carbon dioxide-signal connector 109. The information transmitted includes temperature data and three-dimensional position data from the transesophageal catheter 110 as well as carbon dioxide trigger data. The information is utilized by the microprocessor 112 to control/augment the output of the system.

It is important to note that all electronics and electrical connections are protected in a manner suitable for use in an operating or procedure theater. These precautions are necessary to prevent any interaction between an oxygen source and an electrical spark. In addition, all components are preferably manufactured for medical grade usage.

A system in accordance with the present disclosure preferably comprises a catheter with an anchoring mechanism, such as an inflatable balloon, for fixing the catheter in place in the esophagus to prevent the catheter from moving during the ablation procedure. The catheter has a reversibly deployable needle that advances a short distance from the end of a catheter and locks in that position. The catheter may include a sensor that allows its position to be identified on a mapping device such as the CARTO® 3. The catheter also comprises a user actuatable valve, button, knob or any suitable device that allows for user-mediated delivery of carbon dioxide. The catheter may be connected directly to a small pressurized canister of carbon dioxide with a regulator or to an electronic gas delivery system that: 1) controls the rate and volume of carbon dioxide that can be delivered over the course of the procedure; and 2) for safety, makes it impossible to accidentally hook the device to a gas other than carbon dioxide. The catheter may also comprise a custom combination gas delivery line and signal cable that connects to an electronic gas delivery system to allow for user-mediated delivery of carbon dioxide as well as for the monitoring of temperature sensor data. The catheter may also comprise a wireless communication system (such as Bluetooth) to connect with the electronic gas delivery system. Alternative exemplary embodiments are also contemplated as described in greater detail subsequently.

More specifically, a catheter for administering carbon dioxide through the esophageal wall as part of the above-described system preferably has certain attributes. The catheter has an integrated stopcock to allow for inflation and deflation of a balloon at the distal aspect of the catheter. In an alternative exemplary embodiment, the catheter may comprise an integral sterile carbon dioxide canister to decrease the setup time and make it easier to utilize. The catheter should preferably have the right handling characteristics and column strength to allow for precise navigation of and positioning at the desired point in the esophagus. The catheter comprises a sliding mechanism to allow for precise, controlled deployment of the needle through the esophageal wall. In one exemplary embodiment, the needle assembly may comprise a 25-gauge needle with sufficient radiopacity for visualization under fluoroscopy. The needle may be made of several materials (i.e. stainless steel, nitinol, peek, and other materials) and may be coated or plated with additional materials to increase its visibility under fluoroscopy (i.e. gold or platinum), to increase its strength, and/or reduce the ability for the needle to transfer bacteria from the esophagus to mediastinal tissues (i.e. with antibiotic coatings such as silver or other compounds).

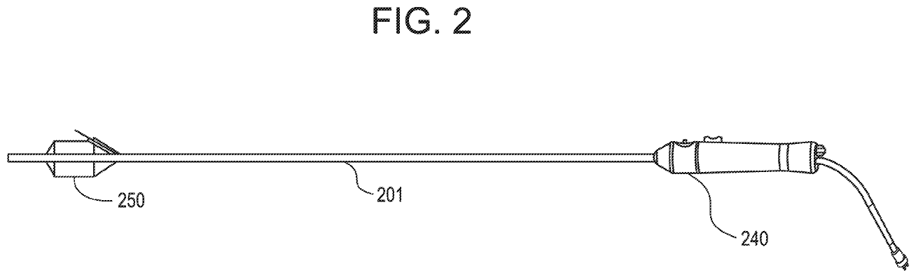

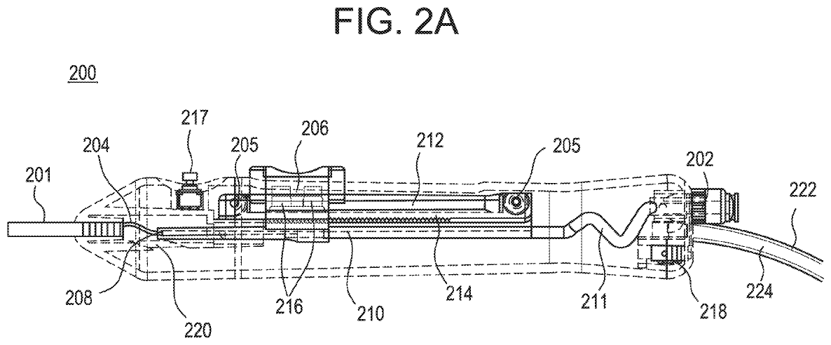

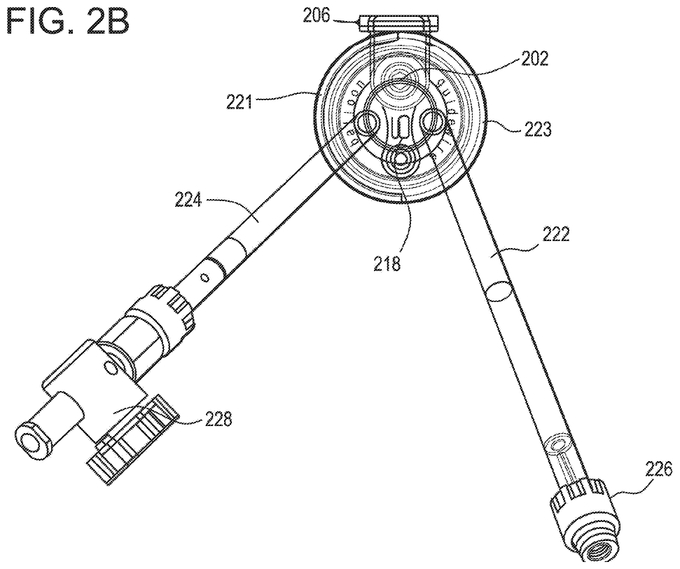

FIG. 2 is a diagrammatic representation of a transesophageal catheter assembly in accordance with an exemplary embodiment of the present disclosure. The transesophageal catheter assembly 200 comprises a proximal end or handle 240 and a distal end/anchoring mechanism assembly 250 interconnected via a shaft 201. Each of the components is described in greater detail subsequently.

Referring to FIGS. 2A and 2B, there is illustrated, in two views, a diagrammatic representation of the proximal end of an exemplary catheter that may be utilized for interventional procedures in accordance with the present disclosure. This device hereinafter will be referred to as a transesophageal catheter, and its various components may be introduced into the esophagus via a variety of methods including an endotracheal device or through a nasogastric tube. The exemplary catheter 200 comprises an elongate body having a proximal end/handle 240 and a distal end/anchoring mechanism assembly 250. The exemplary catheter 200 comprises an ergonomic handle 240 connected to a high-torque, braided shaft 201, which as set forth above connects the handle 240 to the distal end 250. The braided shaft 201 is fixed to the distal end portion of the handle 240.

The carbon dioxide supply or pressurized canister (FIG. 1) is connected to the transesophageal catheter 200 at a male luer connection 202. As set forth above, unique connectors may be utilized to prevent connection to a different gas supply. In addition, this connection point may be utilized to connect any suitable means for flushing the system as described in greater detail subsequently. The luer connection 202 may also be utilized to introduce fluids for any number of purposes, including the delivery of contrast agents for fluoroscopic visualization or the delivery of a hydrogel protective layer to protect the esophagus from thermal damage. Any number of suitable hydrogel protective layers may be utilized and injected through the transesophageal catheter 200. The transesophageal catheter 200 and all of its associated internal components and the high-torque braided shaft 201 are rotationally fixed together to work as a unitary structure. Any suitable means may be utilized to make the connection, including adhesives and welding. With this configuration, rotation of the catheter handle 240 facilitates transmission of torque down the catheter shaft 201 to the anchoring balloon at the distal end 250 of the catheter (not shown in this figure), as described in detail subsequently, to facilitate proper rotational alignment of an injection needle 204 during a procedure. The injection needle 204 is inserted and attached to a needle slider mechanism 206 at connection point 208. The needle slider mechanism 206 may comprise any suitable material. In the exemplary embodiment, the needle slider mechanism 206 comprises a polymeric material and is bonded to the injection needle 204 utilizing any suitable means such that an unobstructed flow of carbon dioxide may be achieved while allowing the components to act as a unitary structure. The injection needle 204 may comprise any suitable biocompatible material, including any hypotube materials currently in use in catheters. The needle slider mechanism 206 allows for advancement and withdrawal of the injection needle 204. Coil tubing 210 is inserted and attached to the needle slider mechanism 206 and exits the handle 240 and terminates at the male luer connection 202. The male luer connection 202 is the connection point with the source of carbon dioxide gas, and carbon dioxide gas travels from the male luer connection 202 through the coil tubing 210 through the needle slider mechanism 206 and through the injection needle 204 into periesophageal tissue. The coil tubing 210 is in a coiled state under rest and allows for the translation of the needle slider mechanism 206 forwards and backwards without breaking the continuity of the carbon dioxide delivery line. The coil tubing 210 is illustrated in the coiled state at 211 as it is connected to the male luer connection 202. The needle slider mechanism 206 is free to translate forwards or backwards to facilitate advancement and withdrawal of the injection needle 204 and is constrained by a rail component 212. The rail component 212 is secured to the handle 240 via screws 205 or other suitable means, for example, pin connectors. Depressing the needle slider mechanism 206 disengages the teeth on the slider with the teeth 214 on the rail component 212. Once released, the teeth on the needle slider mechanism 206 engage with the teeth 214 on the rail component 212 to secure the position of the injection needle 204 in place once the desired tissue penetration depth has been reached. The needle slider mechanism 206 has two spring loaded ball bearings 216 that ride on the surface of the rail component 212 and provide an upward force that maintains the engagement of the teeth on the needle slider mechanism 206 with the teeth 214 on the rail component 212. A carbon dioxide delivery button 217 allows for user-mediated actuation of carbon dioxide delivery and is electrically connected to an electrical connection fitting 218 at the proximal end of the handle 240. Any other suitable electrical connection fitting may be utilized, and the delivery button may comprise any type of user-friendly mechanism. The electrical connection fitting 218 is connected along with the male luer connection 202 to the carbon dioxide delivery system. The guidewire and balloon inflation tubing exit the braided shaft 201 and are directed towards the lower half of the handle 240 via the guiding feature 220. Guidewire tubing 222 and balloon inflation tubing 224 exit the proximal end of the handle 240 as shown in FIG. 2B.

FIG. 2B illustrates the proximal end of the handle 240 and its multitude of connections. The handle 240 comprises a left handle half 221 and a right handle half 223, which can be secured via several manufacturing techniques including ultrasonic welding, glue, or mechanical fixations such as screws. The male luer connection 202 is attached to the carbon dioxide source as set forth above. The electrical connection fitting 218 allows for triggering of the delivery of carbon dioxide gas from the electronic delivery system described above with respect to FIG. 1. This connection may also be used to continuously transfer temperature sensor data from sensors at the distal end of the catheter to the electronic carbon dioxide delivery system. The guidewire tubing 222 exits the handle 240 and terminates at the male luer connection 226. The male luer connection 226 allows for the attachment of a syringe to purge the guidewire tubing lumen with fluid for lubricity as set forth above. A guidewire may be inserted in to the tip of the transesophageal catheter (not shown) and exits therefrom at the male luer connection 226. The guidewire tubing 222 is designated by the text “Guidewire” appearing on the right handle half 223. The balloon inflation line tubing 224 is designated by the text “Balloon” appearing on the left handle half 221 and terminates at the stopcock connection 228. The stopcock connection 228 allows for the attachment of a syringe to inflate and deflate the balloon at the distal end of the transesophageal catheter 200.

The distal end or region of the exemplary transesophageal catheter 200 is continuous with the proximal end or region described herein; however, for ease of explanation as it relates to the present disclosure, the description and drawings are given independently. This basic transesophageal catheter structure may be utilized for any number of interventional procedures, including the introduction and use of a transesophageal catheter for the delivery of carbon dioxide. A detailed description of the distal portion of the transesophageal catheter of the present disclosure, as stated above, is given subsequently.

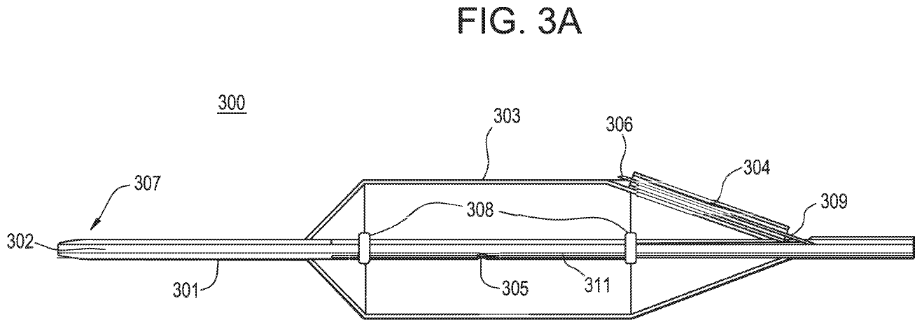

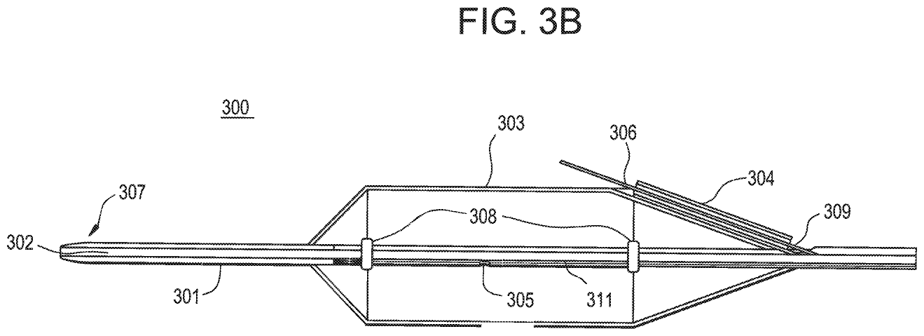

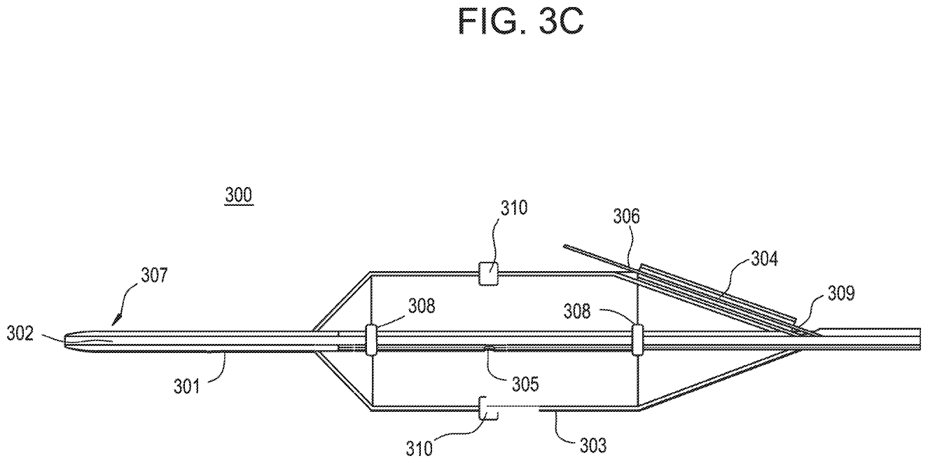

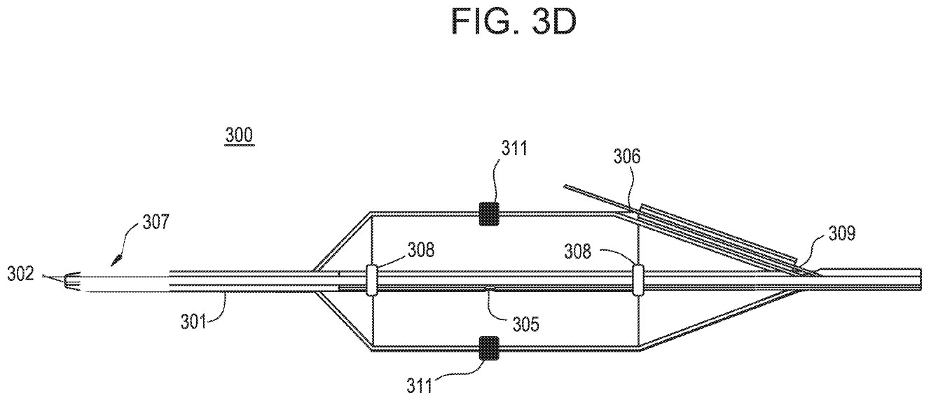

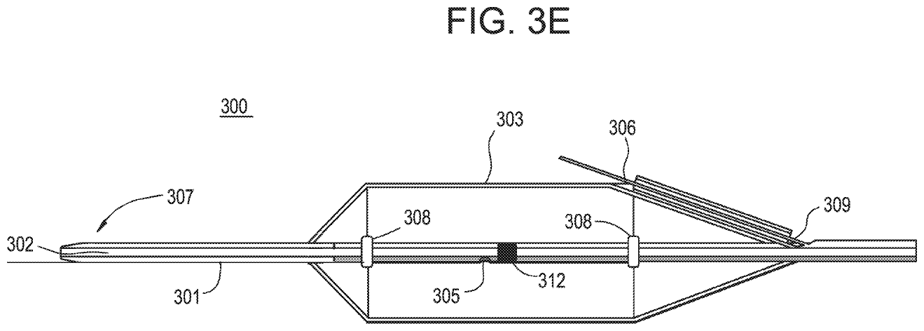

FIGS. 3A and 3B are diagrammatic representations of the distal portion of an exemplary transesophageal catheter in accordance with the present disclosure. FIG. 3A is a first sectional or cutaway view of the distal region of the transesophageal catheter 300. The transesophageal catheter shaft 301 comprises a tubular structure in which a guidewire lumen 302 is coaxially positioned. Attached to the distal end of the transesophageal catheter shaft 301 is a flexible, atraumatic tapered catheter tip 307. The tapered catheter tip 307 functions to guide the transesophageal catheter 300 through the curvature of the oral passageway to the esophagus without causing damage to the surrounding tissue structures. A rapid exchange tip may also be used, which allows for the catheter to ride along the axis of a guidewire without requiring a dedicated lumen that extends through the entire aspect of the catheter shaft. An inflatable balloon 303 is bonded to the transesophageal catheter 300 and functions to anchor the transesophageal catheter 300 in place during injection needle 306 puncture and carbon dioxide insufflation. A protective needle sleeve 304 is affixed to the outer surface of the proximal conic section of the inflatable balloon 303 such that the sharp tip of the injection needle 306 is fully contained and protected to prevent unintentional damage of the esophagus during device introduction. To ensure that the inflatable balloon 303 and the transesophageal catheter shaft 301 move and operate as a unitary structure, the inflatable balloon 303 is permanently bonded to the transesophageal catheter shaft 301 by any suitable means. In one exemplary embodiment, a UV curable adhesive is utilized. The transesophageal catheter shaft 301 may comprise any suitably rigid, biocompatible material that may be navigated through a tortuous path to the esophagus. Standard catheter materials may be utilized. Metallic material, for example, stainless steel, or polymeric materials may be utilized. The inflatable balloon 303 may comprise and suitable biocompatible material that can be repeatedly inflated and deflated. The compliant nature of the balloon allows the user to inflate the inflatable balloon 303 until it reaches the equivalent internal diameter of the esophagus, thereby making the device agnostic to variations in esophageal anatomy (esophageal diameter, curvature, longitudinal variation, and the like). Other non-compliant balloon materials may be used and be precisely sized to the patient's esophageal anatomy. Balloon inflation and deflation is accomplished utilizing techniques known in the catheter art, for example, with room air, and enters the balloon 303 via port 305 which is simply an opening in a balloon inflation line 311. In the exemplary embodiment, the transesophageal catheter shaft 301 comprises braided stainless steel with a PEBAX outer sheath. The aspect of the catheter shaft residing within the inflatable balloon 303 and the needle lumen also feature radiopaque marker bands 308 for fluoroscopic visualization. The radiopaque marker bands 308 may be formed from any suitable material, for example, platinum, tantalum, gold or with surface coatings such as barium sulfate, and bonded to the transesophageal catheter shaft 301 utilizing any suitable means. The radiopaque marker bands 308 are positioned and bonded on the outer surface of the catheter shaft 301.

In operation and prior to needle deployment, illustrated in FIG. 3B, the transesophageal catheter 300 is positioned in-line with the cardiac silhouette. The inflatable balloon 303 is inflated until it contacts and induces tension in the esophageal wall in order to maintain catheter position during needle deployment and carbon dioxide insufflation. The transesophageal catheter 300 can be rotated while the inflatable balloon 303 is inflated to rotationally align the trajectory of the injection needle 306 to the lateral aspect of the patient's esophagus. As set forth above, twisting or rotation of the catheter handle (element 240 in FIGS. 2, 2A and 2B) by the physician facilitates transmission of torque through the transesophageal catheter shaft 301.

FIG. 3B is a sectional or cutaway view of the distal region of the transesophageal catheter 300 in which the translatable injection needle 306 has been deployed. In the exemplary embodiment, the injection needle 306 comprises surgical steel, but may also comprise any other suitable metallic materials, including nitinol and highly radiopaque materials in alternative embodiments. Contrast solution may be delivered through the injection needle 306 to confirm proper advancement through the esophageal wall. Carbon dioxide is then delivered through the injection needle 306 into the fibro-fatty tissue that separates the heart wall from the esophagus until the ablation procedure is completed. Upon completion, the injection needle 306 is retracted, the inflatable balloon 303 is deflated and the transesophageal catheter 300 may be removed. The injection needle 306 exits the protective needle sleeve 304 which resides coaxially within the transesophageal catheter shaft 301 at exit point 309. The transesophageal catheter 300 does not in any way interfere with the ablation procedure.

In accordance with an alternate exemplary embodiment, FIG. 3C illustrates a sectional or cutaway view of the distal region of the transesophageal catheter 300 in which temperature sensors have been embedded or bonded to the surface of the inflatable balloon 303. Other sensors may also be utilized, including pressure, electromagnetic, and impedance. The temperature sensors 310 may monitor, measure, and transmit temperature data of the esophageal wall during the ablation. This data would then be transmitted to the electrical connection 218 on the handle 240 as shown in FIG. 2A. Temperature data may be monitored and recorded by the electrical carbon dioxide delivery system as illustrated in FIG. 1. The microprocessor 112 of the system 100 may include software which utilizes the data from the sensors 310 to provide feedback control as well as for data collection. The sensors 310 may comprise any suitable means for sensing the temperature proximate the ablation site, including simple thermocouples.

In accordance with yet another alternate exemplary embodiment, FIG. 3D illustrates a sectional or cutaway view of the distal region of the transesophageal catheter 300 in which one or more anti-slip grips 311 are bonded or embedded to the external surface of the inflatable balloon 303 in the form of ribs, spikes, pyramids, bumps, curettes, villi or similar protrusions. In addition, one or more radiopaque markers or materials, for example, barium sulfate or radiopaque metal markers, may be attached to or embedded within the inflatable balloon 303 as described above, or attached to or affixed to the one or more anti-slip grips 311 to aid the user in the orientation of the device to the patient's anatomy to aim the injection needle 306.

In accordance with still yet another alternate exemplary embodiment, FIG. 3E illustrates a sectional or cutaway view of the distal region of the transesophageal catheter 300 in which a three-dimensional position sensor 312 may be positioned within and/or on the inflatable balloon 303. This sensor 312 may be utilized to locate the tip of the transesophageal catheter 300 in space relative to the surrounding anatomy as well as to measure insufflation. By working in combination with the Biosense Webster CARTO® 3 system, the distance between the sensor/probe in the heart and the sensor 312 in the catheter tip can be measured and monitored to determine changes in position thereby indicating the amount of insufflation. The Carto® 3 System, available from Biosense® Webster, Inc. a Johnson & Johnson Company, is configured to guide the physicians in their placement of the ablation catheter by verifying the ablation catheter are positioned correctly, in real-time, as they are advanced into the patient. The CARTO® 3 system, particularly its navigation features, are set forth in U.S. Pat. Nos. 6,400,981; 6,650,927; 6,690,963; 6,716,166; 6,788,967; 7,090,639; 7,517,318; 7,536,218; 7,604,601; 7,681,579; 7,684,850; 7,735,349; 7,756,576; 7,831,076; 7,848,787; 7,848,789; 7,869,865; 8,075,486; 8,320,711; 8,359,092; 8,456,182; 8,478,379; 8,478,383; 8,532,738; 8,676,305; 8,870,779; 9,023,027; 9,460,563; 9,498,147, all of which are incorporated by reference herein.

Although the distal portion and the proximal portion of the transesophageal catheter is shown in different illustrations for ease of explanation, the two portions form a continuous structure.

As set forth above, the needle is advanced through the wall of the esophagus into the fibro-fatty tissue or periesophageal compartment to deliver a controlled dose of carbon dioxide to expand the tissue and create an insulation layer during an ablation procedure. In the preferred embodiment, the delivery of carbon dioxide is continuous during ablation rather than through discrete delivery so as to safely maintain tissue expansion. Upon completion of the procedure, the needle may be retracted into the protective sleeve as explained above. In order to precisely deliver the carbon dioxide, the system may employ one or more methodologies to determine the deployment depth of the needle without the need for direct visual confirmation. It is important to note that visual confirmation would be a viable alternative but involve additional complexities.

The needle in any of the exemplary embodiments set forth herein, including those by which the device is puncturing through the esophagus is made of stainless steel; however, a variety of other materials may be used as well. Furthermore, coatings applied to the needle may be used to increase its lubricity, for example, polytetrafluoroethylene or PTFE, to aid in esophageal puncture and/or with antibacterial agents to prevent infection. Anti-adhesive surface coatings using concepts of surface chemistry and functionality including ions and polymer coats may be used. The needle surface may be coated with bactericidal substances such as Chitosan—vancomycin and silver. Nanotopographic surface modifications may also be used as either anti-adhesives or bactericidal features. Furthermore, a radiopaque plating (for example, gold or platinum) can be applied to the needle to aid in fluoroscopic visualization of the needle.

It is also important to note that the balloon utilized in the above-described exemplary embodiments may comprise any suitable type of catheter delivered balloon and are both inflated and deflated in the standard manner.

The balloon 303 may have an integrated additional lumen, that acts as the conduit for the needle or needle lumen. As the balloon 303 is inflated, it points the needle lumen at a certain angle with respect to the esophageal wall, irrespective of the patient-specific anatomy of the esophagus. This angled approach prevents accidental puncture of adjacent organs or structures. The angle can be set by changing the angle of the balloon wall. The injection needle may be deployed proximal or distal to the balloon. In a preferred exemplary embodiment, the injection needle is deployed proximal to the balloon 303. In addition, the balloon 303 is configured to make and maintain contact with the esophagus mucosa.

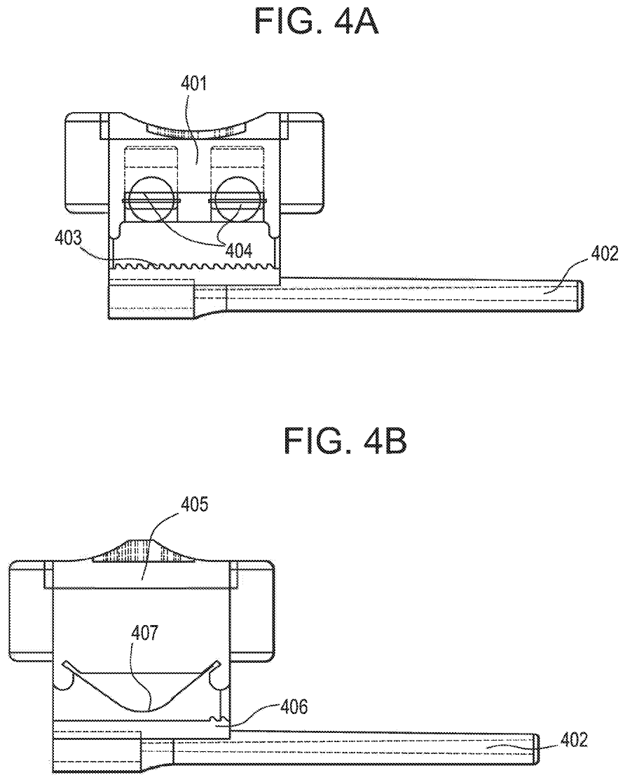

FIGS. 4A and 4B are detailed diagrammatic representations of the needle slider mechanism (206 in FIG. 2A) of an exemplary transesophageal catheter in accordance with the present disclosure. FIG. 4A is a diagrammatic representation of a needle ball-bearing slider mechanism 400. The ball-bearing slider 401 is connected with the injection needle at 402. Depressing the ball-bearing slider 401 disengages the teeth 403 and allows for translation of the slider 401. Once released, the teeth 403 on the ball-bearing slider 401 engage with teeth on the rail insert as described herein (207 in FIG. 2A). The spring-loaded bearings 404 in the ball-bearing needle slider 401 ride on the surface of the rail insert. The spring-loaded bearings 404 push the slider 401 upwards, engaging the teeth 403 on the slider 401 with the teeth on the rail (207 in FIG. 2A).

In accordance with an alternate exemplary embodiment, FIG. 4B is a diagrammatic representation of a needle spring slider mechanism. The spring slider 405 is connected with the needle at 402. Depressing the spring slider 405 disengages the teeth 406 and allows for translation of the slider. Once released, the teeth 406 on the spring slider engage with teeth on the rail insert. The spring steel segment 407 in the spring slider 405 rides on the surface of the rail insert. The spring steel segment 407 pushes the slider 405 upwards, engaging the teeth 406 on the slider 405 with the teeth on the rail (207 in FIG. 2A). Alternatively, the slider could use compression springs, torsion springs, extension springs, magnets shaped metal or polymer that acts as a spring mechanism.

In accordance with alternate exemplary embodiments, the present disclosure may comprise anchoring mechanisms other than balloons.

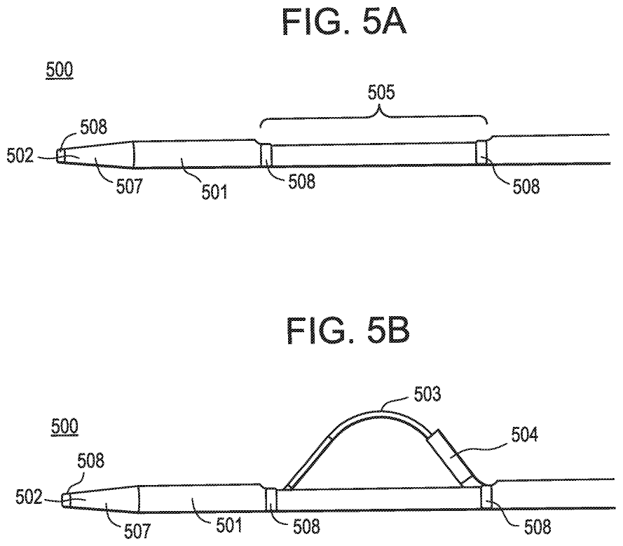

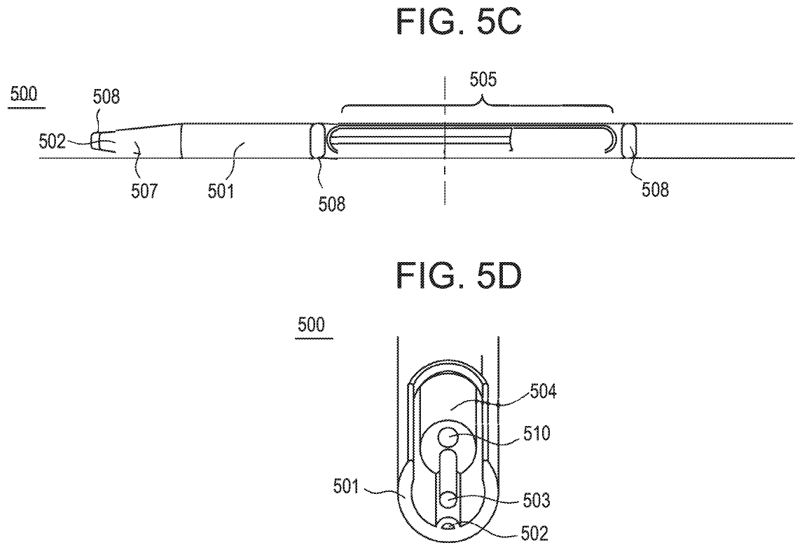

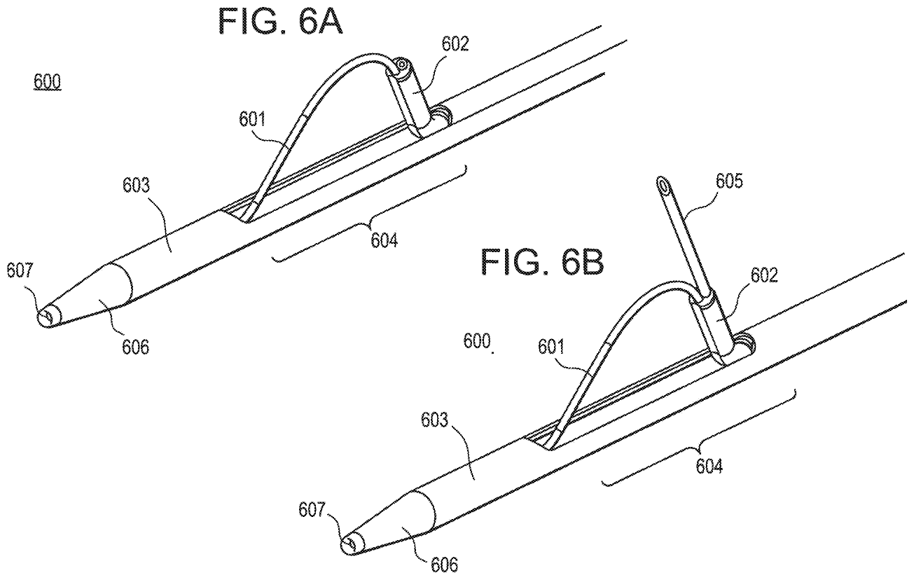

Referring to FIGS. 5A through 5D, there are diagrammatic representations of an alternate exemplary embodiment of the distal portion of an exemplary transesophageal catheter in accordance with the present disclosure. FIG. 5A is a view of the distal region of the transesophageal catheter 500. The exemplary catheter may be utilized for interventional procedures in accordance with the present disclosure. The exemplary transesophageal catheter 500 comprises an elongate body having a proximal end and a distal end. The exemplary transesophageal catheter 500 comprises an ergonomic handle connected to a high-torque, braided shaft 501. The braided catheter shaft 501 is fixed to the device handle 240 (shown in FIG. 2). The transesophageal catheter shaft 501 comprises a tubular structure in which a guidewire lumen 502 is positioned. Attached to the distal end of the catheter shaft 501 is a flexible, atraumatic tapered catheter tip 507. The tapered catheter tip 507 functions to guide the transesophageal catheter 500 through the curvature of the oral passageway to the esophagus without causing damage to the surrounding tissue structures. The catheter shaft 501 may comprise any suitably rigid, biocompatible material that may be navigated through a tortuous path to the esophagus. Standard catheter materials may be utilized. Metallic material, for example, stainless steel, or polymeric materials may be utilized. In the exemplary embodiment, the catheter shaft comprises 501 braided stainless steel with a PEBAX outer sheath. The distal aspect of the catheter shaft 501 also features radiopaque marker bands 508 for fluoroscopic visualization. The radiopaque marker bands 508 may be formed from any suitable material, for example, platinum and bonded to the catheter shaft 501 utilizing any suitable means. Additional materials are described above with respect to previous exemplary embodiments. The radiopaque marker bands 508 are positioned and bonded on the outer surface of the catheter shaft 501. A cutout on the catheter creates an opening 505 for the deployment of a nitinol wire anchor 503, inner catheter 504, and injection needle 605 (shown in FIG. 6B). The inner catheter 504 houses the needle 605 and nitinol wire 503 during device introduction into the esophagus. When the nitinol wire 503 is advanced it induces inflection of the inner catheter 504 and the needle 605 is pulled towards the esophageal wall. The deflectable nitinol wire 503 is bonded to the catheter and functions to anchor the transesophageal catheter in place during needle puncture and carbon dioxide insufflation. This wire arrangement is utilized in place of the balloon as described above. The terminal end of the deflectable nitinol wire 503 is affixed to the catheter shaft 501 so that as the nitinol wire 503 is advanced distally, the nitinol wire deflects outward from a coaxial alignment with the catheter shaft to engage with the esophageal wall. It is important to note that any suitable material that may be repeatedly deflected may be utilized, including both metallic materials as well as polymeric materials. Furthermore, the distal end of the nitinol wire could also be pulled to create the desired deflection. The nitinol wire can be pre-treated to deflect in a particular direction or shape.

In place of a wire, ribbon or other geometrical profiles can be used to minimize esophageal lacerations and tears. The mechanism of inducing deflection of the nitinol wire 503 allows the user to induce deflection until the wire reaches the equivalent internal diameter of the esophagus, thereby making the device agnostic to variations in esophageal anatomy (esophageal diameter, curvature, longitudinal variation, and the like).

FIG. 5D is a sectional or cutaway view of the distal region of the exemplary transesophageal catheter 500 illustrated in FIG. 5C, which is a top view of the devices illustrated in FIGS. 5A and 5B, in accordance with the present disclosure. FIG. 5D is a diagrammatic representation of the catheter shaft 501, the inner catheter 504, the guidewire lumen 502, the deflectable nitinol wire 503, and the needle lumen 510 of the inner catheter 504 in which the injection needle 605 resides and is free to translate forwards and backwards to puncture the wall of the esophagus for the delivery of carbon dioxide gas into periesophageal tissues.

In operation and prior to needle deployment, illustrated in FIG. 6A, the transesophageal catheter 600 is positioned in-line with the cardiac silhouette. The nitinol wire 601 is advanced, causing outward deflection, until it makes contact with and induces tension in the esophageal wall in order to maintain catheter position during needle deployment and carbon dioxide insufflation. The transesophageal catheter 600 can be rotated while the nitinol wire is deflected to rotationally align the trajectory of the needle to the lateral aspect of the patient's esophagus. Twisting or rotating the catheter handle by the physician facilitates transmission of torque through the catheter shaft 603.

The apex of the deflectable nitinol wire 601 curve is in contact with the wall of the esophagus and induces tension in the esophageal wall, anchoring the transesophageal catheter 600 in place. The deflection of the wire 601 also deflects the inner catheter 602 from a coaxial alignment to allow for needle puncture of the esophagus. Opening 604 in the transesophageal catheter 600 allows for deflection of the nitinol wire 601 and needle advancement. The transesophageal catheter 600 includes a tapered tip 606 which is flexible and atraumatic. Opening 607 provides a lumen for guidewire insertion.

FIG. 6B is a sectional view of the distal region of the transesophageal catheter 600 in which the translatable injection needle 605 has been deployed. In the exemplary embodiment, the needle 605 comprises surgical steel, but may also comprise any other suitable metallic materials, including nitinol and highly radiopaque materials in alternative embodiments. Contrast solution may be delivered through the injection needle 605 to confirm proper advancement through the esophageal wall. Carbon dioxide is then delivered through the injection needle 605 the fibro-fatty tissue that separates the posterior left atrium wall from the esophagus until the ablation procedure is completed. Upon completion, the needle 605 is retracted, the nitinol wire 601 is retracted and the transesophageal catheter may be removed. The transesophageal catheter 600 does not in any way interfere with the ablation procedure.

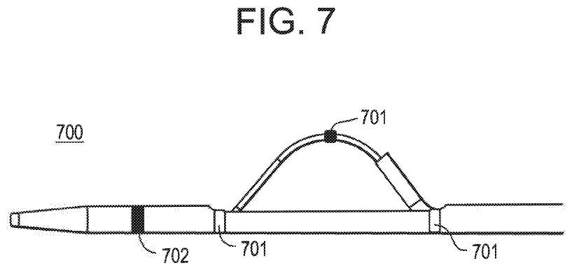

FIG. 7 is a diagrammatic representation of yet another alternate exemplary embodiment of the distal portion of an exemplary transesophageal catheter 700 in accordance with the present disclosure. FIG. 7 is a sectional or cutaway view of the distal region of the transesophageal catheter 700 in which temperature sensors 701 have been embedded or bonded to the surface of the catheter shaft. The temperature sensors 701 may monitor, measure, and transmit temperature data of the esophageal wall during the ablation. Other sensors may also be utilized as set forth above. This data would then be transmitted to the electrical connection on the handle. Temperature data may be monitored and recorded by the electrical carbon dioxide delivery system 100 illustrated in FIG. 1. A three-dimensional position sensor 702 may be positioned within the catheter shaft. This sensor 702 may be utilized to locate the tip of the transesophageal catheter in space relative to the surrounding anatomy as well as to measure insufflation. By working in combination with the Biosense Webster CARTO® 3 system, the distance between the sensor/probe in the heart and the sensor 702 in the catheter tip can be measured and monitored to determine changes in position thereby indicating the amount of insufflation as described above. In addition, there might be radiopaque markers attached to the nitinol wire or the inner catheter for positioning

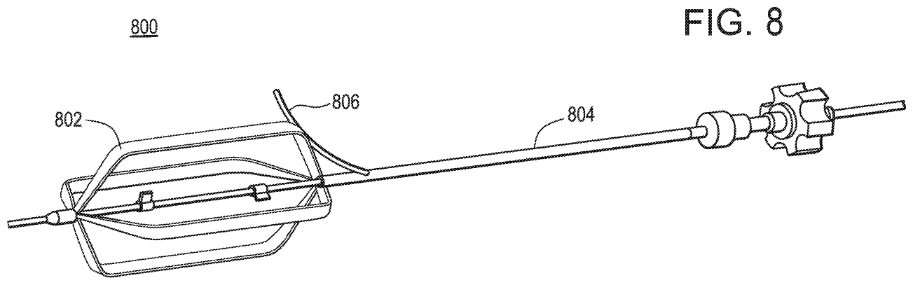

FIG. 8 is an alternative exemplary transesophageal catheter 800 with an expanding nitinol cage anchoring mechanism 802 comprising a heat-set basket in accordance with the present disclosure. This catheter 800 comprises a two-lumen catheter with a protective sheath. The outer sheath prevents the needle 806 point or heat-set basket from damaging the esophagus during navigation to the target insufflation site.

The interior catheter has two lumens, one for guidewire navigation, and a second for needle advancement. The interior catheter also has a heat-set basket that can collapse to fit inside the protective sheath. When the sheath is pulled back, the heat-set basket expands to assume its set shape. The needle 806 is deflected, through attachment to the basket, to facilitate the approach angle of the tip with respect to esophageal wall. The needle 806 is advanced through the lumen of the esophagus into fibro-fatty tissue to deliver a controlled dose of CO₂. The needle 806 can be retracted back into the lumen and the basket can be retracted back into the sheath after insufflation has been achieved. The basket may have anti-slip grips on its external surface in the form of ribs, spikes, pyramids, bumps, villi, or similar protrusions. Radiopaque materials may be embedded on the cage wires, for example barium sulfate or some other suitable metal, to orient the user in properly aiming the needle. The anti-slip grips and radiopaque markers may be incorporated into the same embedded unit.

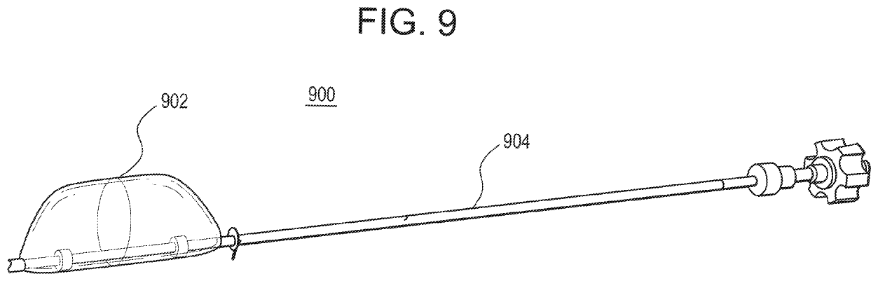

FIG. 9 is an alternative exemplary transesophageal catheter 900 with an expanding asymmetrical balloon anchoring mechanism 902 in accordance with the present disclosure. This catheter comprises a three-lumen catheter 904 with a protective sheath. The outer sheath prevents the needle point from damaging the esophagus during navigation to the target insufflation site. The interior catheter has three lumens, one for guidewire navigation, a second for balloon inflation, and a third for needle advancement. The needle is advanced through the lumen of the esophagus via inflation of the asymmetric balloon. The balloon may have anti-slip grips on its external surface in the form of ribs, spikes, pyramids, bumps, villi, or similar protrusions. Radiopaque materials may be embedded on the surface of the balloon, for example barium sulfate or some other suitable metal, to orient the user in properly aiming the needle. The anti-slip grips and radiopaque markers may be incorporated into the same embedded unit.

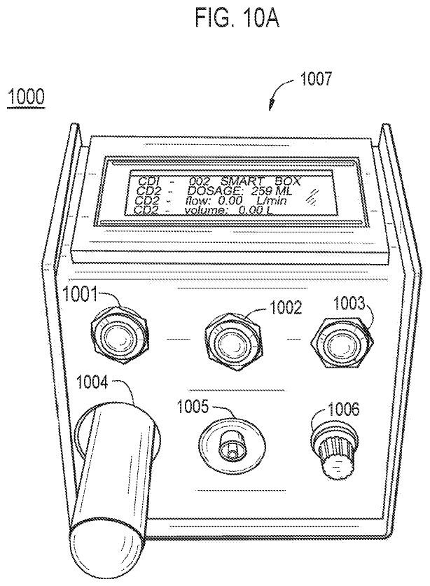

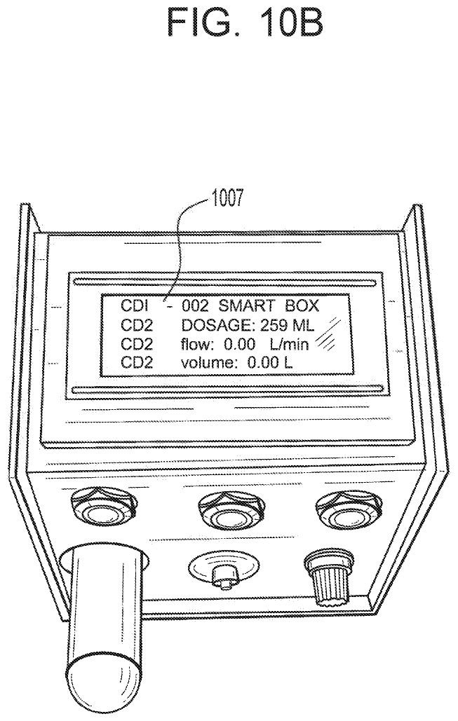

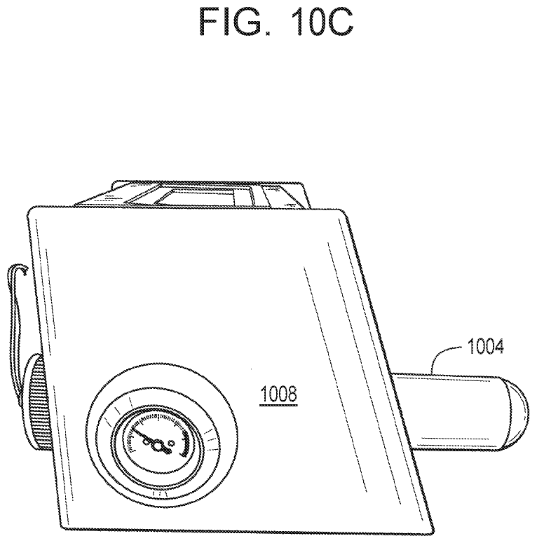

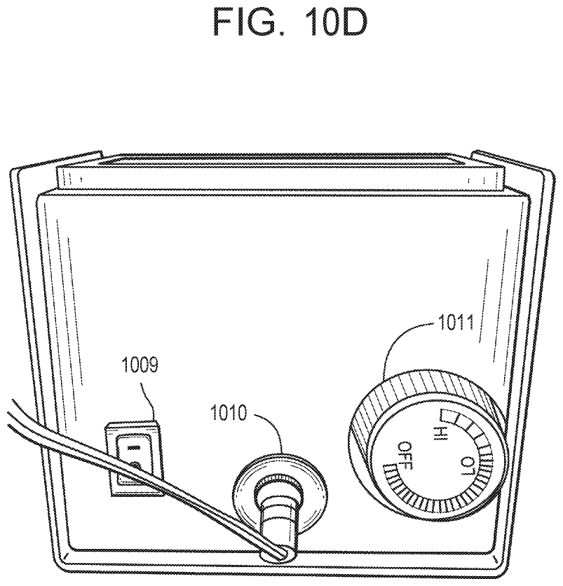

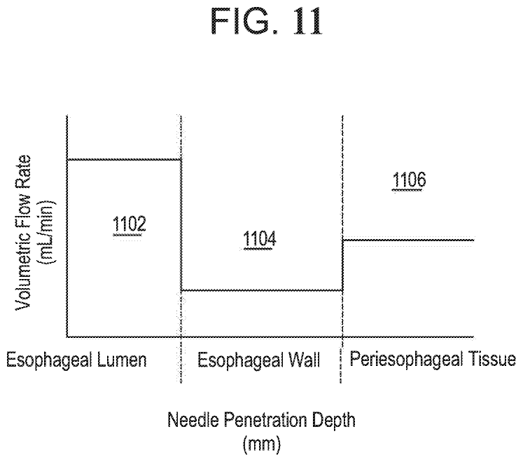

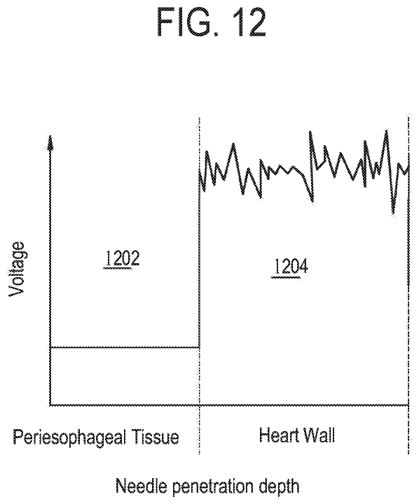

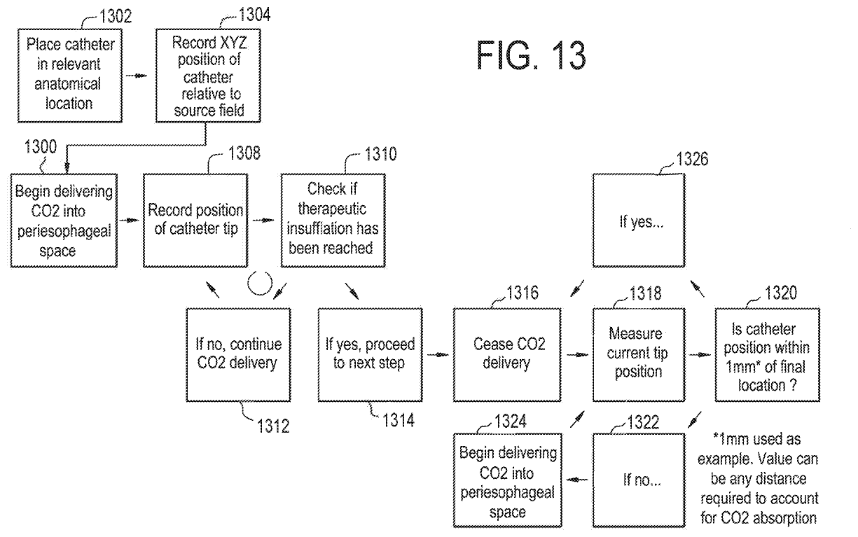

FIG. 10A is a diagrammatic representation of a user interface 1000 of the fluid delivery system (e.g., gas delivery system 100) illustrated in FIG. 1. The pre-set volume push button 1001 allows for the delivery of a user-designated volume of carbon dioxide gas to the transesophageal catheter 110. The cancel/reset button 1002 allows the user to cease the delivery of a preset volume of carbon dioxide. Holding down the cancel/reset button 1002 resets the measurement of the total volume of gas delivered, which is displayed on the LCD display 1007. The on/off push button 1003 allows for user-mediated delivery of carbon dioxide gas. As long as the on/off push button 1003 is depressed, the system will continuously deliver carbon dioxide gas until the button is released. The carbon dioxide gas canister 1004 is inserted into an opening in the case of the gas delivery system 100 and screws into the pressure regulator. Other suitable connections are possible. The transesophageal catheter 110 is attached to the user interface 1000 of the gas delivery system 100 at the CO2-Signal connector 1005. The potentiometer 1006 allows for the user to select a desired volume of gas to be delivered after pressing the pre-set volume push button 1001. The selected volume is displayed on the LCD display 1007. It is important to note that the user interface 1000 is illustrated as a hardware device with buttons and knobs; however, other configurations are possible, including touch screen control.

FIG. 10B is a detailed diagrammatic representation of the exemplary LCD 1007 display of the gas delivery system 100. The LCD display continuously displays the user-set volume of carbon dioxide to be delivered, the real-time volumetric flow rate of gas being delivered, as well as the total volume delivered.

FIG. 10C is a diagrammatic representation of a pressure regulator dial 1008, which is visible to the user to verify the pressure of the gas in the canister 1004. Once again, any suitable display may be utilized to indicate the pressure in the gas cannister 1004.

FIG. 10D is a diagrammatic representation of the power switch 1009, power supply input 1010, and pressure regulator adjustment knob 1011 of the user interface 1000 of the gas supply system 100.

In one exemplary method, the flow rate of the carbon dioxide exiting the needle may be monitored to determine the resistance to flow. The esophageal lumen, the esophageal mucosa and the fibro-fatty tissue all have different resistivity to gas flow. Accordingly, the physician may simply determine in which tissue layer the needle tip resides by referencing a tissue layer flow rate characterization chart. In an alternative embodiment, the microprocessor 112 (FIG. 1) may be programmed with the flow resistivity of the various tissues or media in the body and receiving feedback from the flowmeter 108 as to the flow rate exiting from the needle, automatically generate an alert via some suitable signal to be displayed or an audible signal that indicates that the proper location for the needle has been achieved. The flowmeter may be utilized to measure the flow resistance at the needle tip and provide feedback directly to the physician or through the microprocessor 112 rather than flowmeter 108.

FIG. 11 is a graphical representation of the relative flow rates of carbon dioxide in the different regions/tissues. The vertical axis represents volumetric flow rate and the horizontal axis represents needle penetration depth in millimeters. In the first region 1102 which represents the free space of the esophageal lumen, the flow rate of carbon dioxide is high relative to the other regions as one may expect. In the second region 1104 which represents the esophageal wall, the flow rate of carbon dioxide is significantly lower that the first region 1102 given the density of the esophageal tissue. In the third region 1106 representing the periesophageal tissue, the flow rate of carbon dioxide is higher than in the esophageal wall due to lower tissue density but lower than in the esophageal lumen. By measuring flow rate as the needle progresses, one may determine needle location.

In another exemplary method, the electrical activity of the tissue in which the needle is positioned may be monitored. The myocardium has a distinctly different electrical activity profile than periesophageal fibro-fatty tissue. If misused, the gas delivery system can detect and notify the physician that the needle has been advanced too far and is at risk for puncturing the heart wall by monitoring electrical activity with the needle tip. The physician can thereby determine the point at which the needle has inadvertently contacted the myocardium. In this exemplary embodiment, the needle may be configured to provide feedback to a stand-alone sensing circuit or one that is part of the microprocessor. The sensing circuit may be configured to measure the electrical activity, for example, voltage/potential and/or resistance/impedance. As in the previously described embodiment, this information may be routed through the microprocessor 112 which will automatically make the determination or to any suitable device for altering the physician.

FIG. 12 is a graphical representation of voltage, or potential, on the vertical axis, versus needle penetration depth, on the horizontal axis. As illustrated, in the first region 1202 corresponding to periesophageal tissue, the voltage sensed by the needle is steady-state and low. In the second region 1204 corresponding to the heart wall, the electrical activity is not steady-state and at a higher potential than the first region.

In both exemplary embodiments, real-time monitoring of needle location is achieved without the need for direct visualization.

Referring now to FIG. 13, there is illustrated a simple flow diagram of the overall process. In step 1302, the physician places the transesophageal catheter in the desired anatomical location, for example, the esophagus proximate the left atrium of the heart. Once the transesophageal catheter is in position, the coordinates, in three-dimensional space, of the transesophageal catheter is recorded relative to the source field, step 1304. Once these initial coordinates are recorded, carbon dioxide is delivered into the periesophageal space, step 1306. In step 1308, the position of the catheter tip is recorded, once again in three-dimensional space. At step 1310, a check is performed to see if therapeutic insufflation has been achieved. If therapeutic insufflation has not been achieved, additional carbon dioxide is delivered, step 1312. If therapeutic insufflation has been achieved, step 1314, then the delivery of carbon dioxide is stopped, step 1316. At step 1318, a measurement of the catheter tip in three-dimensional space is repeated. At step 1320, a determination is made as to whether the catheter tip is within a certain distance from the final desired position, for example, 1 mm. This value is utilized as an example. The actual value may be any distance required to account for carbon dioxide absorption. If the catheter tip is not within a predetermined distance from the final desired position, step 1322, then proceed to step 1324 wherein additional carbon dioxide is delivered into the periesophageal space wherein step 1318 is repeated. If the catheter tip is within the predetermined position, step 1326, carbon dioxide delivery is stopped, step 1316. The logic and calculation are performed via the microprocessor 112 of the system.

The proximity of pulmonary veins to the esophagus is a concern in the electrophysiology community. When ablating the former, the risk of damage to the latter is high, and it limits the effectiveness of treatment. The present disclosure creates physical and thermal separation between the two structures by injecting bio-absorbable CO2 in the fibro fatty tissue in between these structures. The novel method generates separation exploiting the anatomical proximity of the first generation of airway branches (from the trachea) to the pulmonary veins. A device in the form of a balloon catheter with an injection needle, or of a dedicated endotracheal tube with dedicated needle lumen may be inserted in the patient's upper airways. Under visualization (e.g. fluoroscopy) the delivery mechanism (e.g. needle) is advanced through the airway toward the pulmonary veins and CO2 delivered.

A method may comprise: delivering a hollow body into the heart; advancing at least a portion of the hollow body through the heart wall; delivering a volume of fluid through the hollow body to create separation between the esophagus and the heart wall; and removing the hollow body after the delivery of fluid.

A method may comprise: delivering a hollow body into the esophagus; advancing at least a portion of the hollow body through the esophageal wall; delivering a volume of fluid through the hollow body to create separation between the esophagus and the heart wall; and removing the hollow body after the delivery of fluid.

A method may comprise: advancing at least a portion of a hollow body percutaneously into the patient's body; delivering a volume of fluid through the hollow body to create separation between the esophagus and the heart wall; and removing the hollow body after the delivery of fluid.

A method may comprise: delivering a hollow body into the airway; advancing at least a portion of the hollow body through the wall of the trachea; delivering a volume of fluid through the hollow body to create separation between the esophagus and the heart wall; and removing the hollow body after the delivery of fluid.

Although shown and described in what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated but should be constructed to cohere with all modifications that may fall within the scope of the appended claims.

What is claimed is:

1. A system for the prevention of esophageal damage during cardiac ablation, the system comprising:
a fluid supply configured for the delivery of a fluid;
a hollow body in fluid-communication with the fluid supply, the hollow body configured to be disposed between a first biological surface and a second biological surface; and
a mechanism configured to control delivery of fluid from the fluid supply and through the hollow body to create separation between the first biological surface and the second biological surface.

2. The system according to claim 1, wherein the hollow body comprises a needle configured to pierce one or more of the first biological surface or the second biological surface.

3. The system according to claim 1, wherein one or more of the first biological surface or the second biological surface comprises tissue.

4. The system according to claim 1, wherein one or more of the first biological surface or the second biological surface comprises esophageal tissue.

5. The system according to claim 1, wherein one or more of the first biological surface or the second biological surface comprises cardiac tissue.

6. The system according to claim 1, wherein the first biological surface comprises at least a portion of an esophagus and the second biological surface comprises at least a portion of a wall of a heart.

7. The system according to claim 1, wherein the hollow body is generally tubular.

8. The system according to claim 1, wherein the hollow body further comprises an electrical connection to the fluid supply.

9. The system according to claim 1, wherein the hollow body further comprises at least one sensor configured to measure at least a temperature, flow rate, flow volume, pressure, and impedance of the fluid flowing through the hollow body.

10. The system according to claim 1, wherein the hollow body further comprises an anchoring mechanism.

11. The system according to claim 10, wherein the anchoring mechanism is disposed adjacent an end of the hollow body.

12. The system according to claim 10, wherein the anchoring mechanism is configured to releasably secure the hollow body to a surface.

13. The system according to claim 10, wherein the anchoring mechanism is configured to releasably secure the hollow body to one or more of the first biological surface or the second biological surface.

14. The system according to claim 1, wherein the delivered fluid comprises carbon dioxide.

15. The system according to claim 1, wherein the delivered fluid comprises a hydrogel material.

16. The system according to claim 1, further comprising a position mechanism configured to determine the position of the hollow body relative to anatomical structures.

17. The system according to claim 16, wherein the position mechanism comprises a volumetric fluid flow meter configured to determine the flow rate of fluid through the hollow body.

18. The system according to claim 16, wherein the position mechanism comprises a pressure gauge.

19. The system according to claim 16, wherein the position mechanism comprises a pressure sensor.

20. The system according to claim 16, wherein the position mechanism comprises an electric potential sensor.

21. The system according to claim 16, wherein the position mechanism comprises at least one radiopaque marker affixed to hollow body.

22. The system according to claim 16, wherein the position mechanism comprises at least one position sensor.

23. The system according to claim 1, wherein the mechanism for the controlled delivery of fluid comprises:
at least one actuatable valve; and
a controller configured to control the operation of the at least one actuatable valve based upon user input.

24. The system according to claim 23, wherein the mechanism for controlled delivery of fluid further comprises an adjustable pressure gauge.

25. The system according to claim 23, wherein the mechanism for controlled delivery of fluid further comprises a fluid flow meter.

26. The system according to claim 23, wherein the mechanism for controlled delivery of fluid further comprises a pressure sensor.

27. The system according to claim 23, wherein the controller comprises a depressible button.

28. A system for to creating separation between biological surfaces, the system comprising:
a hollow body configured to access a target location;
a control element configured to control the delivery of a fluid through the hollow body;
a sensor device configured to measure a parameter of the fluid flowing through the hollow body, wherein the parameter is used to determine at least an environment of the hollow body such that the hollow body may be moved to the target location in response to one or more of the parameter or changes to the parameter.

29. The system according to claim 28, wherein the fluid comprises carbon dioxide gas.

30. The system according to claim 28, wherein the fluid comprises a hydrogel material.

31. The system according to claim 28, wherein the control element comprises a hard-wired electronic switch.

32. The system according to claim 28, wherein the control element comprises a wireless electronic switch.

33. The system according to claim 28, wherein the control element comprises a potentiometer dial.

34. The system according to claim 28, wherein the sensor comprises at least one temperature sensor.

35. The system according to claim 28, wherein the sensor comprises at least one impedance sensor.

36. The system according to claim 28, wherein the sensor comprises at least one pressure sensor.

37. The system according to claim 28, wherein the sensor comprises at least one volumetric flow meter.

38. The system according to claim 28, further comprising a display, wherein an indication of the parameter is communicated to a user via the display.

39. The system according to claim 28, wherein the parameter comprises at least the total volume of fluid delivered through the hollow body.

40. The system according to claim 28, wherein the parameter comprises at least the total volume of fluid delivered through the hollow body within a selected period of time.

41. The system according to claim 28, wherein the parameter comprises at least the real-time flow rate a fluid through the hollow body.

42. The system according to claim 28, wherein the parameter comprises at least temperature readings.

43. The system according to claim 28, wherein the parameter comprises at least pressure readings.

44. The system according to claim 28, wherein the parameter comprises at least impedance readings.

45. The system according to claim 28, further comprising a communication mechanism configured to communicate to a user via an audible alarm.

46. The system according to claim 45, wherein the audible alarm is activated when temperature measurements approach or reach a predetermined level.

47. The system according to claim 45, wherein the audible alarm is activated when flow rate measurements approach or reach a predetermined level.

48. A system for to creating separation between biological surfaces, the system comprising:
a hollow body configured to access a target location;
a control element configured to actuate the delivery of a fluid through the hollow body;
a sensor device configured to measure a parameter of the fluid flowing through the hollow body, wherein the parameter is used to actuate a flow of the fluid through the hollow body in response to one or more of the parameter or changes to the parameter.

49. The system according to claim 48, wherein the fluid comprises carbon dioxide gas.

50. The system according to claim 48, wherein the fluid comprises a hydrogel material.

51. The system according to claim 48, wherein the control element comprises a hard-wired electronic switch.

52. The system according to claim 48, wherein the control element comprises a wireless electronic switch.

53. The system according to claim 48, wherein the control element comprises a potentiometer dial.

54. The system according to claim 48, wherein the sensor comprises at least one temperature sensor.

55. The system according to claim 48, wherein the sensor comprises at least one impedance sensor.

56. The system according to claim 48, wherein the sensor comprises at least one pressure sensor.

57. The system according to claim 48, wherein the sensor comprises at least one volumetric flow meter.

58. The system according to claim 48, wherein the sensor comprises at least one position sensor.

59. The system according to claim 48, further comprising a display, wherein an indication of the parameter is communicated to a user via the display.

60. The system according to claim 48, wherein the parameter comprises at least the total volume of fluid delivered through the hollow body.

61. The system according to claim 48, wherein the parameter comprises at least the total volume of fluid delivered through the hollow body within a selected period of time.

62. The system according to claim 48, wherein the parameter comprises at least the real-time flow rate a fluid through the hollow body.

63. The system according to claim 48, wherein the parameter comprises at least temperature readings.

64. The system according to claim 48, wherein the parameter comprises at least pressure readings.

65. The system according to claim 48, wherein the parameter comprises at least impedance readings.

66. The system according to claim 48, wherein the parameter comprises at least position readings.

67. The system according to claim 48, further comprising a communication mechanism configured to communicate to a user via an audible alarm.

68. The system according to claim 67, wherein the audible alarm is activated when temperature measurements approach or reach a predetermined level.

69. The system according to claim 67, wherein the audible alarm is activated when flow rate measurements approach or reach a predetermined level.

70. The system according to claim 67, wherein the audible alarm is activated when a volume of fluid flowing through the hollow body approaches or reaches a predetermined level.
